SEQUENCE LISTING (4 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 4 aa, Artificial Sequence
  The first four amino acids for the B-domain of human Factor VIII sequence
SFSQ

SEQ ID NO: 3 - protein, 10 aa, Artificial Sequence
  The last ten amino acids for the B-domain of human Factor VIII sequence
NPPVLKRHQR

SEQ ID NO: 4 - protein, 1457 aa, Artificial Sequence
  Derived from human Factor VIII sequence
MQIELSTCFFLCLLRFCFSATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

(19) United States
(12) Patent Application Publication
(10) Pub. No.: US 2011/0112022 A1
(43) Pub. Date: May 12, 2011

(54) Factor VIII Muteins with Reduced Immonugenicity

(75) Inventors: Fred Jullien Aswad, San Francisco, CA (US); Richard Harkins, Alameda, CA (US); Hsiao-Lai Liu, Martinez, CA (US); John E. Murphy, Berkeley, CA (US); Faye Wu, Pinole, CA (US); Ying Zhu, Alamo, CA (US)

(73) Assignee: BAYER HEALTHCARE LLC, Tarrytown, NY (US)

(21) Appl. No.: 13/001,360

(22) PCT Filed: Jun. 25, 2009
(86) PCT No.: PCT/US09/48680

§ 371 Date: Dec. 23, 2010

Related U.S. Application Data
(60) Provisional application No. 61/075,494, filed on Jun. 25, 2008.

(51) Int. Cl. A61K 38/37; C07K 14/755; C07H 21/00; C12N 15/63; C12N 5/071; A61K 31/7088

(52) U.S. Cl. 514/14.1; 530/383; 536/23.5; 435/320.1; 435/325; 514/44 R

(57) ABSTRACT

The invention relates to modified Factor VIII molecules with reduced N-linked glycosylation and reduced immunogenicity. The invention also relates to methods of using modified Factor VIII molecules, for example, to treat patients afflicted with hemophilia.

This application claims benefit of U.S. Provisional Application Ser. No. 61/075,494; filed on Jun. 25, 2008, the contents of which are incorporated herein by reference in their entirety.

### FIELD OF THE INVENTION

The invention relates generally to mutated Factor VIII molecules (Factor VIII muteins) having mutations in certain non-capped N-linked glycosylation sites. These muteins exhibit reduced uptake by antigen-presenting dendritic cells and reduced immunogenicity when used therapeutically.

### BACKGROUND OF THE INVENTION

Human therapeutic proteins (biologics) isolated from natural sources or synthesized through recombinant methods can induce immune responses when administered to human patients. These immune responses can lead to effects ranging from minor skin irritation to decreased efficacy of the therapeutic drug, and in some instances can cause massive organ failure or death.

Approximately 30% of patients treated with recombinant Factor VIII (rFVIII) exhibit an immune response. Of these patients, about one in three exhibit neutralizing antibodies (nAbs) against Factor VIII (FVIII), and these antibodies can interfere with the efficacy of the FVIII therapy (Ehrenforth, et al., Lancet 339:594-598, 1992; Gringeri, et al., Blood 102:2358-2363, 2003). While high-dose administration of FVIII can reduce the effect of nAbs in this patient population, the compliance burden and costs associated with higher-dosage treatment regimens are undesirable.

The major type of immune eliciting antigen presenting cells (APC) is a dendritic cell (DC). DCs can endocytose proteins via different types of cell-surface receptors. Endocytosis leads to processing of the protein into peptides, loading of individual peptides onto MHC Class II (MHCII) proteins, and display of the peptide MHCII complex on the cell surface (Trombetta, et al., Annu Rev Immunol 23:975-1028, 2005). Recognition of these peptides by T helper cells induces downstream events which can lead to immunogenicity and/or immunotoxicity.

Recent reports suggest that CD206, a mannose specific receptor, plays a role in the uptake of FVIII by APCs. The interaction between FVIII and CD206 leads to endocytosis of the FVIII/CD206 complex and degradation of the rFVIII protein into peptides which are then displayed by MHC class II proteins on the surface of APCs (Dasgupta, et al., Proc Natl Acad Sci USA 104:8965-8970, 2007). CD206 has been shown to recognize a number of different carbohydrate structures (mannose, fucose, and N-acteylglucosamine) with varying affinities (Lee, et al., Science 295:1898-1901, 2002). However, among the members of the mannose receptor family, CD206 appears to have the greatest affinity for mannose. FVIII has been shown to contain both capped (capped by sialyation) and non-capped (non-sialyated) glycosylation sites (Kaufman, et al., J Biol Chem 263:6352-6362, 1988; Medzihradszky, et al., Anal Chem 69:3986-3994, 1997). Non-capped sites terminate with a mannose residue and therefore, are sometimes termed mannose-ending glycosylation sites. Because non-capped glycosylations on FVIII terminate with mannose residues, they could act as recognition sites for CD206.

Potential sites for either capped or non-capped glycosylation occur on the FVIII molecule at N-linked glycosylation sites. N-linked glycosylation occurs on the asparagine residue within the amino acid sequence motif N-X-S/T, where X can be any amino acid except proline. Full-length mature FVIII contains 24 putative N-linked glycosylation sites.

Human FVIII contains the structural domains A1-A2-B-A3-C1-C2 (Thompson, Semin Hematol 29:11-22, 2003). The B-domain of FVIII is dispensable, since B-domain deleted FVIII (BDD) is also effective as a replacement therapy for hemophilia A.

There are 19 putative N-linked glycosylation sites within the B-domain, therefore removal of the complete B-domain leaves 5 residual N-linked glycosylation sites in the BDD FVIII (BDD) at amino acid positions 41, 239, 582, 1810, and 2118. The N-linked glycosylation sites at amino acid positions 239, 1810, and 2118 normally show a higher level of N-linked glycosylation than the sites at amino acid positions 41 and 582.

Production of recombinant proteins with altered glycosylation patterns presents several challenges, including a potential drop in productive yield from recombinant culture and/or decreased activity of the recombinant protein.

The problem of FVIII immunogenicity has been recognized in the art and a number of approaches have been suggested for reducing the immunogenicity of FVIII with the objective of improving its therapeutic efficacy.

Immunogenicity of FVIII can be reduced by conjugation of FVIII to an alcoholic polymer such as polyethylene glycol (PEGylation) (U.S. Pat. No. 4,970,300). U.S. Pat. No. 7,351,688 discloses complexing a therapeutic protein such as FVIII with a binding agent such as a phospholipid. Human/animal FVIII hybrid molecules, wherein certain immunogenic portions of the human FVIII molecule have been replaced with porcine FVIII sequences are described as being less immunogenic in humans than is human FVIII (see, e.g., U.S. Pat. Nos. 5,364,771; 6,180,371; 6,458,563; and 7,012,132). The immunogenicity of FVIII can be reduced by introduction of additional sites for N-linked glycosylation into FVIII epitopes which are known to react with anti-FVIII antibodies (U.S. Pat. No. 6,759,216).

Another strategy which has been proposed is to reduce immunogenicity of FVIII by introducing mutations into areas of the FVIII molecule which bind with anti-FVIII antibodies (see, e.g., U.S. Pat. Nos. 7,211,559; 7,122,634; 7,033,791; 6,770,744; and 6,376,463).

FVIII muteins containing a mutation which introduces a cysteine residue at several amino acid positions in the FVIII molecule including positions 239, 1810, 1812, and 2118, where the introduced cysteine residue provides a site for PEGylation of the FVIII mutein (U.S. Published Patent Application No. 20060115876 A1).

Thus, a therapeutic protein such as FVIII which exhibits reduced uptake by antigen-presenting dendritic cells and reduced immunogenicity would provide a useful treatment for patients in need of FVIII therapy, for example, hemophilia.

### SUMMARY OF THE INVENTION

The present invention provides a recombinant FVIII molecule comprising a mutation within one or more naturally-occurring non-capped, N-linked glycosylation sequence motifs which occur at amino acid positions 41-43, 239-241, 582-584, 1810-1812, and 2118-2120 of a FVIII molecule. In one embodiment, the mutation does not introduce a cysteine residue at amino acid positions 41, 239, 1810, 1812, or 2118. These mutations prevent the site which has been mutated from being glycosylated when the rFVIII molecule is expressed in a glycosylation-competent host cell. In one embodiment, the mutation occurs at one or more of amino acid positions 239-241, 1810-1812, and 2118-2120.

In another embodiment, the FVIII molecule is a B-domain deleted FVIII mutein (BDD mutein). BDD muteins with substitutions in non-capped N-linked glycosylation sites have been found to be expressed recombinantly at relatively high levels and they exhibit activity levels similar to or increased in relation to non-mutated BDD.

In a further embodiment, the invention comprises an isolated nucleic acid that encodes the rFVIII molecules.

In another embodiment, the invention comprises an expression vector comprising the nucleic acid of the invention.

In another embodiment, the invention comprises a glycosylation-competent host cell comprising the expression vector of the invention.

In another embodiment, the invention comprises a cell culture comprising the glycosylation-competent host cell of the invention.

In another embodiment, the invention comprises a pharmaceutical composition comprising the recombinant FVIII molecule of the invention and a pharmaceutically acceptable carrier. This composition can be lyophilized for storage and reconstituted into a liquid for administration, as is conventional in the art.

In yet another embodiment, the invention comprises a method of treating a patient in need of FVIII therapy, which comprises administering to said patient a therapeutically effective amount of the recombinant FVIII molecule of the invention.

### DESCRIPTION OF THE FIGURES

FIG. 1 demonstrates uptake of full-length rFVIII and deglycosylated full-length rFVIII (FVIII Degly) in vitro by dendritic cells (DCs). Before deglycosylation, rFVIII was labeled with fluorescein isothiocyanate (FITC) for detection by FACS analysis. rFVIII was deglycosylated using Endo-F1 for 60 minutes, co-cultured with DCs for 30 minutes, and then washed. Uptake of FVIII and FVIII Degly by DCs was then analyzed by FACS. Uptake of FVIII Degly is shown relative to the uptake of FVIII, where uptake of FVIII is 100%. An unpaired Student's T-test was performed comparing FVIII Degly with FVIII; ** p<0.01 for FVIII.

FIG. 2 shows the activity (2A) and concentration (2B) of a B-domain deleted FVIII (BDD), and three BDD muteins (N239Q, N2118Q, N239Q/N2118Q). The nomenclature used shows the amino acid substitution at the indicated position, for example N239Q indicates substitution of glutamine for asparagine at amino acid position 239 in the molecule. HKB11 cells were separately transfected with BDD mutant constructs encoding N239Q, N2118Q, and N239Q/N2118Q. Following expression of the proteins, conditioned media were assayed for activity by chromogenic assay (2A) and concentration was assayed by ELISA (2B) at 96 hours post-transfection.

FIG. 3 shows uptake of full-length rFVIII, a B-domain deleted FVIII (BDD), and N-glycosylation site BDD single (N2118Q), and double-mutein (N239Q/N2118Q) by dendritic cells (DCs). DCs were co-cultured with FVIII, BDD, BDDN2118Q, or BDD N239Q/N2118Q for 30 minutes at 4° C. (4C) and 37° C. (37C). Cells were then washed, and the concentration (pM) of FVIII, BDD, BDD N2118Q, and BDD N239Q/N2118Q in cell extracts was measured by ELISA. An unpaired Student's T-test was performed comparing N2118Q and N239Q/N2118Q with FVIII and BDD; ** p<0.01 for both FVIII and BDD.

FIG. 4 shows a reduced IFNγ (4A) and proliferative (4B) response of FVIII-specific T-cell clone BO1-4 against N2118Q. Briefly, FVIII, BDD, or N2118Q was incubated with DCs for 24 hours before co-culture with FVIII-specific T-cell clones. IFNγ response was measured by ELISA 24 hours later. Proliferative responses were measured 6 days later by examining 3H-thymidine incorporation. An unpaired Student's T-test was performed comparing N2118Q with FVIII and BDD; ** p<0.01 for both FVIII and BDD.

### DESCRIPTION OF THE INVENTION

It is to be understood that this invention is not limited to the particular methodology, protocols, cell lines, animal species or genera, constructs, and reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural reference unless the context clearly dictates otherwise. Thus, for example, reference to “an amino acid” is a reference to one or more amino acids and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### Definitions

For convenience, the meaning of certain terms and phrases employed in the specification, examples, and appended claims are provided below.

Factor VIII (FVIII) is a glycoprotein synthesized and released into the bloodstream by the liver. Upon activation by thrombin, it dissociates from the complex to interact with other clotting factors in the coagulation cascade, which eventually leads to the formation of a thrombus. Human full-length FVIII has the amino acid sequence of SEQ ID NO:1, although allelic variants are possible. It is to be understood that this definition includes native as well as recombinant forms of FVIII. The terms “mutein” and “variant” when referring to the polypeptides of the application means muteins and variants of the polypeptides which retain biological function or activity.

As used herein, B domain deleted FVIII (BDD) is characterized by having the amino acid sequence which contains a deletion of all but 14 amino acids of the B-domain of FVIII. The first 4 amino acids of the B-domain (SFSQ, SEQ ID NO:2) are linked to the 10 last residues of the B-domain (NPPVLKRHQR, SEQ ID NO:3) (Lind, et al, Eur. J. Biochem. 232:19-27, 1995). The BDD used herein has the amino acid sequence of SEQ ID NO:4. Examples of BDD polypeptides are described in U.S. Published Patent Application No. 20060115876 A1 which is incorporated herein by reference.

A “mutation” as used herein to describe the FVIII molecule means at least one substitution in a nucleic acid encoding an N-linked glycosylation sequence motif which produces at least one amino acid difference in the encoded mutein and which removes the glycosylation motif and thereby prevents N-linked glycosylation from occurring at that motif in the mutated molecule. The term “mutation” also includes the changed motif resulting from the mutated nucleic acid.

In the examples that follow, the muteins are named in a manner conventional in the art. The convention for naming mutants is based on the amino acid sequence for the mature, full length FVIII as provided in SEQ ID NO:1. For example, the mutation N239Q indicates the asparagine at amino acid position 239 has been changed to glutamine.

As an example, the FVIII muteins may contain conservative substitutions of amino acids. A conservative substitution is recognized in the art as a substitution of one amino acid for another amino acid that has similar properties and include, for example, the changes of alanine to serine; arginine to lysine; asparagine to glutamine or histidine; aspartate to glutamate; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The single letter abbreviation for a particular amino acid, its corresponding amino acid, and three letter abbreviation are as follows: A, alanine (Ala); C, cysteine (Cys); D, aspartic acid (Asp); E, glutamic acid (Glu); F, phenylalanine (Phe); G, glycine (Gly); H, histidine (His); I, isoleucine (IIe); K, lysine (Lys); L, leucine (Leu); M, methionine (Met); N, asparagine (Asn); P, proline (Pro); Q, glutamine (Gin); R, arginine (Arg); S, serine (Ser); T, threonine (Thr); V, valine (Val); W, tryptophan (Trp); Y, tyrosine (Tyr); and norleucine (Nle).

As used herein, protein and polypeptide are synonyms.

Glycosylation of polypeptides is typically either N-linked or O-linked. N-linked refers to the attachment of a carbohydrate moiety to the side chain of an asparagine residue. The tripeptide sequences Asn-X-Ser and Asn-X-Thr (“N-X-S/T”), where X is any amino acid except proline, are the recognition sequences for enzymatic attachment of the carbohydrate moiety to the Asn side chain. Thus, the presence of either of these tripeptide sequences (or motifs) in a polypeptide creates a potential N-linked glycosylation site.

Since these sequence motifs (N-X-S/T) are necessary for N-linked glycosylation, several different types of mutation can prevent N-linked glycosylation at these sites. These mutations include, for example, substitution of the asparagine residue (N) by another residue, substitution of the second residue (X) with proline, or substitution of the third residue (S/T) with any amino acid except serine or threonine.

Certain substitutions in N-linked glycosylation sequence motifs would not prevent glycosylation within the motif, for example, a substitution of serine for threonine at the third position. A skilled artisan can determine readily which substitutions would, or would not, prevent glycosylation from occurring at the mutated glycosylation site.

In one embodiment, a mutation may be a substitution of the asparagine residue at position one of the motif (N-X-S/T) by a residue of similar amino acid such as glutamine. By replacing an asparagine residue with a glutamine residue in an N-linked glycosylation site, it is possible to inhibit glycosylation at these sites while generally maintaining the polarity and hydropathy of the native molecule at these positions.

In another embodiment, the mutation is a substitution of an asparagine with a glutamine residue at position 239 (N239Q). In another embodiment, the mutation is a substitution of an asparagine with a glutamine residue at position 2118 (N2118Q). In a further embodiment, the mutation is a substitution of an asparagine with a glutamine residue at positions 239 and 2118 (N239Q/N2118Q).

The rFVIII molecule of the invention can be either a full-length FVIII molecule or a functional variant thereof, provided that the molecule contains a mutation which prevents glycosylation at one of the sequence motifs occurring at amino acid positions 41-43, 239-241, 582-584, 1810-1812, and 2118-2120 of a FVIII molecule. The FVIII molecule may optionally be mutated at other amino acid positions, providing that activity is retained. The mutations in the FVIII molecule should not introduce a cysteine residue into the mutein, since cysteine residues can result in the formation of undesired reactions including cysteine bonds.

In one embodiment, the FVIII molecule is a B-domain deleted variant (BDD) in which the B domain has been deleted in part or entirely. The BDD may retain one or more of the N-linked glycosylation sites found in the B domain (see, e.g., U.S. Pat. No. 4,868,112 and EP294910). In another embodiment, the BDD lacks essentially all of the B-domain. By “essentially all” is meant that at least the region encompassing all of the known glycosylation sites within the B-domain. An example of this embodiment of BDD is a BDD FVIII molecule having an amino acid sequence in which all but 14 amino acids of the B-domain of FVIII have been deleted. The first 4 amino acids of the B-domain are linked to the 10 last residues of the B-domain (see, e.g., U.S. Published Application No. 20060115876). Alternatively, the BDD can lack the entire B-domain (see, e.g., U.S. Pat. No. 6,130,203).

Amino acid sequence alteration may be accomplished by a variety of techniques, for example, by modifying the corresponding nucleic acid sequence by site-specific mutagenesis. Techniques for site-specific mutagenesis are well known in the art and are described in, for example, Zoller et al., (DNA 3:479-488, 1984) or Horton, et al., (Gene 77:61-68, 1989, pp. 61-68). For example, the FVIII nucleotide sequence can be mutated using the Stratagene cQuickChange™ II site-directed mutagenesis kit (Stratagene Corporation, La Jolla, Calif.). Successful mutagenesis can be confirmed by DNA sequencing, and appropriate fragments containing the mutation can be transferred into the FVIII backbone in a mammalian expression vector that confers resistance to, for example, Hygromycin B (Hyg B). After transfer, the mutations can again be sequence-confirmed. Thus, using the nucleotide and amino acid sequences of FVIII, one may introduce the alteration(s) of choice. Likewise, procedures for preparing a DNA construct using polymerase chain reaction using specific primers are well known to persons skilled in the art (see, e.g., PCR Protocols, 1990, Academic Press, San Diego, Calif., USA).

The nucleic acid construct encoding FVIII may also be prepared synthetically by established standard methods, for example, the phosphoramidite method described by Beaucage, et al., (Gene Amplif. Anal. 3:1-26, 1983). According to the phosphoamidite method, oligonucleotides are synthesized, for example, in an automatic DNA synthesizer, purified, annealed, ligated, and cloned in suitable vectors. The DNA sequences encoding FVIII may also be prepared by polymerase chain reaction using specific primers, for example, as described in U.S. Pat. No. 4,683,202; or Saiki, et al., (Science 239:487-491, 1988). Furthermore, the nucleic acid construct may be of mixed synthetic and genomic, mixed synthetic and cDNA, or mixed genomic and cDNA origin prepared by ligating fragments of synthetic, genomic, or cDNA origin (as appropriate), corresponding to various parts of the entire nucleic acid construct, in accordance with standard techniques.

The DNA sequences encoding FVIII may be inserted into a recombinant vector using recombinant DNA procedures. The choice of vector will often depend on the host cell into which the vector is to be introduced. The vector may be an autonomously replicating vector or an integrating vector. An autonomously replicating vector exists as an extrachromosomal entity and its replication is independent of chromosomal replication, for example, a plasmid. An integrating vector is a vector that integrates into the host cell genome and replicates together with the chromosome(s) into which it has been integrated.

The vector may be an expression vector in which the DNA sequence encoding the modified FVIII is operably linked to additional segments required for transcription, translation, or processing of the DNA, such as promoters, terminators, and polyadenylation sites. In general, the expression vector may be derived from plasmid or viral DNA, or may contain elements of both. The term “operably linked” indicates that the segments are arranged so that they function in concert for their intended purposes, for example, transcription initiates in a promoter and proceeds through the DNA sequence coding for the polypeptide.

Expression vectors for use in expressing FVIII may comprise a promoter capable of directing the transcription of a cloned gene or cDNA. The promoter may be any DNA sequence that shows transcriptional activity in the host cell of choice and may be derived from genes encoding proteins either homologous or heterologous to the host cell. Examples of promoters for directing the transcription of the DNA in mammalian cells are, for example, the SV40 promoter (Subramani, et al., Mol. Cell Biol. 1:854-864, 1981), the MT-I (metallothionein gene) promoter (Palmiter, et al., Science 222:809-814, 1983), the CMV promoter (Boshart, et al., Cell 41:521-530, 1985), or the adenovirus 2 major late promoter (Kaufman et al., Mol. Cell Biol, 2:1304-1319, 1982).

The DNA sequences encoding FVIII may also, if necessary, be operably connected to a suitable terminator (see e.g., Palmiter, et al., Science 222:809-814, 1983; Alber et al., J. Mol. Appl. Gen. 1:419-434, 1982; McKnight, et al., EMBO J. 4:2093-2099, 1985). The expression vectors may also contain a polyadenylation signal located downstream of the insertion site. Polyadenylation signals include the early or late polyadenylation signal from SV40, the polyadenylation signal from the adenovirus 5 EIb region, the human growth hormone gene terminator (DeNoto, et al., Nucl. Acids Res. 9:3719-3730, 1981). The expression vectors may also include enhancer sequences, such as the SV40 enhancer.

The procedures used to ligate the DNA sequences coding for FVIII or FVIII muteins, the promoter, the terminator, and optionally other sequences, and to insert them into suitable vectors containing the information necessary for replication, are well known to persons skilled in the art (see, e.g., Sambrook et al., Molecular Cloning: A Laboratory Manual, Cold Spring Harbor, N.Y., 1989).

Suitable expression vectors containing the nucleic acid encoding the FVIII mutein may be introduced into glycosylation competent cells. FVIII expression can then be assayed by ELISA and activity can be assayed using a conventional assay such as the Coatest chromogenic assay (diaPharma, West Chester, Ohio).

Methods of transfecting mammalian cells and expressing DNA sequences introduced into the cells are described in, for example, Kaufman, et al., (J. Mol. Biol. 159:601-621, 1982); Southern, et al., (J. Mol. Appl. Genet. 1:327-341, 1982); Loyter, et al., (Proc. Natl. Acad. Sci. USA 79:422-426, 1982); Wigler, et al., (Cell 14:725-731, 1978); Corsaro, et al., (Somatic Cell Genetics 7:603-616, 1981), Graham, et al., (Virology 52:456-467, 1973); and Neumann, et al., (EMBO J. 1:841-845, 1982). Cloned DNA sequences may be introduced into cultured mammalian cells by, for example, lipofection, DEAE-dextran-mediated transfection, microinjection, protoplast fusion, calcium phosphate precipitation, retroviral delivery, electroporation, sonoporation, laser irradiation, magnetofection, natural transformation, and biolistic transformation (see, e.g., Mehier-Humbert, et al., Adv. Drug Deliv. Rev. 57:733-753, 2005). To identify and select cells that express the exogenous DNA, a gene that confers a selectable phenotype (a selectable marker) is generally introduced into cells along with the gene or cDNA of interest. Selectable markers include, for example, genes that confer resistance to drugs such as neomycin, puromycin, hygromycin (Hygromycin B, Hyg B), and methotrexate. The selectable marker may be an amplifiable selectable marker, which permits the amplification of the marker and the exogenous DNA when the sequences are linked. Exemplary amplifiable selectable markers include dihydrofolate reductase (DHFR) and adenosine deaminase. It is within the purview of one skilled in the art to choose suitable selectable markers (see, e.g., U.S. Pat. No. 5,238,820).

After cells have been transfected with DNA, they are grown in an appropriate growth medium to express the gene of interest. As used herein the term “appropriate growth medium” means a medium containing nutrients and other components required for the growth of cells and the expression of FVIII or FVIII muteins (see, e.g., U.S. Pat. Nos. 5,171,844; 5,422,250; 5,422, 260; 5,576,194; 5,612,213; 5,618,789; 5,804,420; 6,114,146; 6,171825; 6,358,703; 6,780,614; and 7,094,574).

Media generally include, for example, a carbon source, a nitrogen source, essential amino acids, essential sugars, vitamins, salts, phospholipids, protein, and growth factors. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of FVIII or FVIII muteins.

For example, the transfected cells may be placed under selective pressure with 50 μg/mL Hyg B in a growth medium supplemented with 5% FBS. Hyg B-resistant colonies are selected and screened for FVIII expression. The stable transformants are then adapted to a culture medium for recombinant expression. Generation and expression of FVIII muteins is described in several publications (see, e.g., U.S. Published Application No. 20060115876; Kaufman, et al., J Biol Chem 263:6352-6362, 1988; Hironaka, et al., J Biol Chem 267:8012-8020, 1992).

Examples of mammalian cell lines for use in the present invention are the COS-1 (ATCC CRL 1650), baby hamster kidney (BHK), HKB11 cells (Cho, et al., J. Biomed. Sci, 9:631-638, 2002), and HEK-293 (ATCC CRL 1573; Graham, et al., J. Gen. Virol. 36:59-72, 1977) cell lines. In addition, a number of other cell lines may be used within the present invention, including rat Hep I (rat hepatoma; ATCC CRL 1600), rat Hep II (rat hepatoma; ATCC CRL 1548), TCMK-1 (ATCC CCL 139), Hep-G2 (ATCC HB 8065), NCTC 1469 (ATCC CCL 9.1), CHO-K1 (ATCC CCL 61), and CHO-DUKX cells (Urlaub and Chasin, Proc. Natl. Acad. Sci. USA 77:4216-4220, 1980).

Certain cell lines are capable of glycosylating recombinant proteins and are referred to herein as “glycosylation competent” cell lines. One example of a glycosylation competent cell line is HKB11 which is available from American Type Culture Center (ATCC number CRL-12568). Other glycosylation competent cell lines useful in the invention include COS-1, CHO, HEK293, and BHK cells.

A recombinant culture comprising host cells containing a nucleic acid sequence encoding a FVIII mutein is grown under suitable conditions to express and recover the mutein. In one embodiment, the FVIII mutein may expressed in a secreted form by the host cells, recovered from the growth medium, and optionally further purified to produce a pharmaceutical product.

FVIII polypeptides may be recovered from cell culture medium and may then be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation)), extraction (see, e.g., Protein Purification, Janson and Lars Ryden, editors, VCH Publishers, New York, 1989), or various combinations thereof. In an exemplary embodiment, the polypeptides may be purified by affinity chromatography on an anti-FVIII antibody column. Additional purification may be achieved by conventional chemical purification means, such as high performance liquid chromatography. Other methods of purification are known in the art, and may be applied to the purification of the modified FVIII polypeptides (see, e.g., Scopes, R., Protein Purification, Springer-Verlag, N.Y., 1982).

Generally, “purified” shall refer to a protein or peptide composition that has been subjected to fractionation to remove various other components, and which substantially retains its expressed biological activity. Where the term “substantially purified” is used, this designation shall refer to a composition in which the protein or peptide forms the major component of the composition, such as constituting about 50%, about 60%, about 70%, about 80%, about 90%, about 95%, about 99%, or more of the proteins in the composition.

Various methods for quantifying the degree of purification of the polypeptide are known to those of skill in the art. These include, for example, determining the specific activity of an active fraction, or assessing the amount of polypeptides within a fraction by SDS/PAGE analysis. An exemplary method for assessing the purity of a fraction is to calculate the specific activity of the fraction, compare the activity to the specific activity of the initial extract, and to thus calculate the degree of purity, herein assessed by a “-fold purification number.” The actual units used to represent the amount of activity will, of course, be dependent upon the particular assay technique.

Recombinant FVIII can be produced on a commercial scale. Any suitable culture procedure and culture medium may be used to culture the cells in the process of the invention. Suitable culture procedures, conditions, and media are well known in the cell culture art. Batch and continuous fermentation procedures, either suspension and adherent culture, for example, microcarrier culture methods and stirred tank and airlift fermenters may be used as appropriate. Host cells may be cultured in any type of culture equipment such as fermentation vessels. The cells may be cultured as adherent cell cultures or as suspension cell cultures. Equipment for suspension cell culture of cells expressing recombinant protein is familiar to the skilled artisan (see, e.g., U.S. Pat. Nos. 7,294,484; 7,157,276; 6,660,501; and 6,627,426). In general, principles, protocols, equipment, and practical techniques for anchorage-independent suspension cell culture can be found in Chu, et al. (Curr Opin Biotechnol 12:180-7, 2001) and Warnock, et al. (Biotechnol Appl Biochem 45:1-12, 2006).

The culture medium used to culture the cells may comprise various known and available growth media. Either serum supplemented or serum free media may be used. For the production of therapeutic proteins, the medium may be a serum-free and/or protein-free medium (see, e.g., U.S. Pat. Nos. 5,804,420 and 7,094,574; WO 97/05240; and EP 0 872 487).

### Pharmaceutical Compositions

The invention also concerns pharmaceutical compositions for parenteral administration comprising therapeutically effective amounts of the FVIII muteins of the invention and a pharmaceutically acceptable carriers. Pharmaceutically acceptable carriers are substances that may be added to the active ingredient to help formulate or stabilize the preparation and cause no significant adverse toxicological effects to the patient. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Supplementary active ingredients also may be incorporated into the compositions.

The compositions of the present invention include classic pharmaceutical preparations. Administration of these compositions according to the present invention may be via any common route. The pharmaceutical compositions may be introduced into the subject by any conventional method, for example, by intravenous, intradermal, intramuscular, subcutaneous, or transdermal delivery. The treatment may consist of a single dose or a plurality of doses over a period of time.

The pharmaceutical forms, suitable for injectable use, include sterile aqueous solutions or dispersions and sterile powders for the extemporaneous preparation of sterile injectable solutions or dispersions. The form should be sterile and should be fluid to the extent that easy syringability exists. It should be stable under the conditions of manufacture and storage and should be preserved against the contaminating action of microorganisms, such as bacteria and fungi. The carrier may be a solvent or dispersion medium containing, for example, water, ethanol, polyol (e.g., glycerol, propylene glycol, and liquid polyethylene glycol, and the like) sucrose, L-histidine, polysorbate 80, or suitable mixtures thereof, and vegetable oils. The proper fluidity may be maintained, for example, by the use of a coating, such as lecithin, by the maintenance of the required particle size in the case of dispersion, and by the use of surfactants. The prevention of the action of microorganisms may be brought about by various antibacterial an antifungal agents, for example, parabens, chlorobutanol, phenol, sorbic acid, thimerosal, and the like. The injectable compositions may include isotonic agents, for example, sugars or sodium chloride. Prolonged absorption of the injectable compositions may be brought about by the use in the compositions of agents delaying absorption, for example, aluminum monostearate and gelatin.

FVIII pharmaceutical compositions may also include bulking agents, stabilizing agents, buffering agents, surfactants, sodium chloride, calcium salts, and other excipients. These excipients may be chosen to maximize the stability of FVIII in lyophilized preparations and in liquid formulations.

The bulking agents can include, for example, mannitol, glycine, alanine, and hydroxyethyl starch (HES). The stabilizing agents may include sugars such as sucrose, trehalose, and raffinose, sugar alcohols such as sorbitol and glycerol, or amino acids such as arginine.

Buffer agents may be present in these formulations because the FVIII molecule may be adversely affected by changes in pH during lyophilization. The pH may be maintained in the range of between 6 and 8 during lyophilization, for example, at a pH of about 7. The buffering agent can be any physiologically acceptable chemical entity or combination of chemical entities which have the capacity to act as buffers, including histidine, Tris, BIS-Tris propane, 1,4-piperazinediethanesulfonic acid (PIPES), 3-(N-morpholino)propanesulfonic acid (MOPS), 4-(2-hydroxyethyl)-1-piperazineethanesulfonic acid (HEPES), 2-(N-morpholino)ethanesulfonic acid (MES), and N-[carbamoylmethyl]-2-aminoethane-sulfonic acid (ACES).

Sterile injectable solutions may be prepared by incorporating the active compounds (e.g., FVIII muteins) in the required amount in the appropriate solvent with various of the other ingredients enumerated above, as required, followed by filtered sterilization.

Generally, dispersions may be prepared by incorporating the various sterilized active ingredients into a sterile vehicle that contains the basic dispersion medium and the required other ingredients from those enumerated above. In the case of sterile powders for the preparation of sterile injectable solutions, methods of preparation include, for example, vacuum-drying and freeze-drying techniques that yield a powder of the active ingredient plus any additional desired ingredient from a previously sterile-filtered solution thereof.

Upon formulation, solutions may be administered in a manner compatible with the dosage formulation and in such amount as is therapeutically effective. “Therapeutically effective amount” is used herein to refer to the amount of a polypeptide that is needed to provide a desired level of the polypeptide in the bloodstream or in the target tissue. The precise amount will depend upon numerous factors, for example, the particular FVIII mutein, the components and physical characteristics of the therapeutic composition, intended patient population, mode of delivery, individual patient considerations, and the like, and can readily be determined by one skilled in the art, based upon the information provided herein.

The formulations may be easily administered in a variety of dosage forms, such as injectable solutions, and the like. For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous and intraperitoneal administration.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000)

Examples of pharmaceutical compositions of FVIII are disclosed, for example, in U.S. Pat. Nos. 5,047,249, 5,656,289, 5,665,700 5,690,954, 5,733,873, 5,919,766, 5,925,739, 6,835,372, and 7,087,723.

### Methods of Treatment

Based on well known assays used to determine the efficacy for treatment of conditions identified above in mammals, and by comparison of these results with the results of known medicaments that are used to treat these conditions, the effective dosage of the muteins of this invention may readily be determined for treatment of each desired indication. The amount of the active ingredient to be administered in the treatment of one of these conditions can vary widely according to such considerations as the particular polypeptide and dosage unit employed, the mode of administration, the period of treatment, the age and sex of the patient treated, and the nature and extent of the condition treated.

Appropriate dosages may be ascertained through the use of established assays for determining blood clotting levels in conjunction with relevant dose response data. The final dosage regimen may be determined by the attending physician, considering factors that modify the action of drugs, for example, the drug's specific activity, severity of the damage, and the responsiveness of the patient, the age, condition, body weight, sex and diet of the patient, the severity of any infection, time of administration, and other clinical factors.

The compositions described herein may be used to treat any bleeding disorder associated with functional defects of FVIII or deficiencies of FVIII such as altered binding properties of FVIII, genetic defects of FVIII, and a reduced plasma concentration of FVIII. Genetic defects of FVIII comprise, for example, deletions, additions, and/or substitution of bases in the nucleotide sequence encoding FVIII. In one embodiment, the bleeding disorder may be hemophilia. Symptoms of such bleeding disorders include, for example, severe epistaxis, oral mucosal bleeding, hemarthrosis, hematoma, persistent hematuria, gastrointestinal bleeding, retroperitoneal bleeding, tongue/retropharyngeal bleeding, intracranial bleeding, and trauma-associated bleeding.

The compositions of the present invention may be used for prophylactic applications. In some embodiments, FVIII muteins may be administered to a subject susceptible to or otherwise at risk of a disease state or injury to enhance the subject's own coagulative capability. Such an amount may be defined to be a “prophylactically effective dose.” Administration of FVIII muteins for prophylaxis includes situations where a patient suffering from hemophilia is about to undergo surgery and the polypeptide is administered between one to four hours prior to surgery. In addition, the polypeptides are suited for use as a prophylactic against uncontrolled bleeding, optionally in patients not suffering from hemophilia. Thus, for example, the polypeptide may be administered to a patient at risk for uncontrolled bleeding prior to surgery.

In one embodiment of the invention, pharmaceutical compositions of FVIII muteins may be infused into patients intravenously to treat uncontrolled bleeding due to FVIII deficiency (e.g., intraarticular, intracranial, or gastrointestinal hemorrhage) in hemophiliacs.

As an example, the coagulant activity of FVIII in vitro may be used to calculate the dose of FVIII for infusions in human patients (Lusher, et al., New Engl J Med 328:453-459, 1993; Pittman, et al., Blood 79:389-397, 1992; Brinkhous, et al., Proc Natl Acad Sci 82:8752-8755, 1985). In one embodiment, the plasma FVIII level to be achieved in a patient via administration of the FVIII mutein may be in the range of 30-100% of normal.

In another embodiment, the composition may be given intravenously at a dosage in the range from about 5 to 50 units/kg body weight, or in a range of 10-50 units/kg body weight, or at a dosage of 20-40 units/kg body weight. Treatment can take the form of a single intravenous administration of the composition or periodic or continuous administration over an extended period of time, as required. The interval frequency is in the range from about 8 to 24 hours (in severely affected hemophiliacs), and the duration of treatment is in the range from 1 to 10 days or until the bleeding episode is resolved.

The FVIII muteins of the invention may also be expressed in vivo, that is, these muteins may be used for gene therapy. Cells may be engineered with a polynucleotide (DNA or RNA) encoding a FVIII mutein ex vivo and the engineered cells may then be provided to a patient to be treated with the polypeptide. Such methods are well known in the art. For example, cells may be engineered by procedures known in the art by use of a retroviral particle containing RNA encoding for polypeptides of the present invention. The gene to be administered may be isolated and purified using ordinary molecular biology and recombinant DNA techniques within the skill of the art. The isolated gene may then be inserted into an appropriate cloning vector (e.g., adenoviruses, adeno-associated virus (AAV), vaccinia, herpesviruses, baculoviruses and retroviruses, parvovirus, lentivirus, bacteriophages, cosmids, plasmids, fungal vectors). The coding sequences of the gene to be delivered may be operably linked to expression control sequences, such as promoters, enhancers, transcriptional and translational stop sites, and other signal sequences.

Delivery of a therapeutic vector into a patient may be either direct, in which case the patient is directly exposed to the vector or a delivery complex, or indirect, in which case, cells are first transformed with the vector in vitro, then transplanted into the patient. These two approaches are known, respectively, as in vivo and ex vivo gene therapy. For example, the therapeutic vector may be directly administered in vivo by direct injection of naked DNA, or by use of microparticle bombardment (e.g., a gene gun).

Several methods for transferring potentially therapeutic genes to defined cell populations are known (see, e.g., Mulligan, Science 260:926-31, 1993). These methods include, for example: 1) direct gene transfer (see, e.g., Wolff, et al., Science 247:1465-68, 1990); 2) liposome-mediated DNA transfer (see, e.g., Caplen, et al., Nature Med 3:39-46, 1995; Crystal, Nature Med. 1:15-17, 1995; Gao and Huang, Biochem Biophys Res Comm 179:280-85, 1991); 3) retrovirus-mediated DNA transfer (see, e.g., Kay, et al., Science 262:117-19, 1993; Anderson, Science 256:808-13, 1992); 4) DNA virus-mediated DNA transfer. Such DNA viruses include adenoviruses (e.g., Ad-2 or Ad-5 based vectors), herpes viruses (e.g., herpes simplex virus based vectors), and parvoviruses (e.g., adeno-associated virus based vectors, such as AAV-2 based vectors) (see, e.g., Ali, et al., Gene Therapy 1:367-84,1994; U.S. Pat. No. 4,797,368; U.S. Pat. No. 5,139,941). Other methods of gene therapy are described by Goldspiel, et al., (Clin Pharm 12:488-505, 1993); Wu and Wu (Biotherapy 3:87-95, 1991); Tolstoshev (Ann Rev Pharmacol Toxicol 32:573-596, 1993); and Morgan and Anderson, (Ann Rev Biochem 62:191-217, 1993). Methods commonly known in the art of recombinant DNA technology that can be used are described in Ausubel, et al., (Current Protocols in Molecular Biology, John Wiley & Sons, N.Y., 1993); Kriegler, (Gene Transfer and Expression, A Laboratory Manual, Stockton Press, N.Y., 1990); Dracopoli, et al., (Current Protocols in Human Genetics, John Wiley & Sons, N.Y., 1994); and Colosimo, et al., (Biotechniques 29:314-324, 2000).

The choice of a particular vector system for transferring a gene of interest will depend on a variety of factors. The skilled artisan will appreciate that any suitable gene therapy vector encoding polypeptides of the invention can be used in accordance with this embodiment. The techniques for constructing such vectors are known (see, e.g., Anderson, Nature 392:25-30, 1998; Verma and Somia, Nature 389:239-242, 1998). Introduction of the vector to the target site may be accomplished using known techniques.

Suitable gene therapy vectors include one or more promoters. Suitable promoters which may be used include, but are not limited to, viral promoters (e.g., retroviral LTR, SV40 promoter, adenovirus major late promoter, respiratory syncytial virus promoter, B19 parvovirus promoter, and human cytomegalovirus (CMV) promoter described in Miller, et al., Biotechniques 7:980-990, 1989), cellular promoters (e.g., histone, pol III, and β-actin promoters), and inducible promoters (e.g., MMT promoter, metallothionein promoter, and heat shock promoter). The selection of a suitable promoter will be apparent to those skilled in the art from the teachings contained herein.

Retroviruses from which the retroviral plasmid vectors may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, Myeloproliferative Sarcoma Virus, and mammary tumor virus. The retroviral plasmid vector may be used to transduce packaging cell lines to form producer cell lines. Examples of packaging cells which maybe transfected include, but are not limited to, the PE501, PA317, PA12, VT-19-17-H2, and DAN cell lines as described in Miller (Human Gene Therapy, 1:5-14, 1990). The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host. The producer cell line generates infectious retroviral vector particles that include the nucleic acid sequence(s) encoding muteins of the invention. Such retroviral vector particles then may be used, to transduce eukaryotic cells, either in vitro or in vivo. The transduced eukaryotic cells will express the nucleic acid sequence(s) encoding muteins of the invention. Eukaryotic cells that can be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

In one embodiment, the DNA encoding the FVIII muteins of the invention is used in gene therapy for disorders such as hemophilia. According to this embodiment, gene therapy with DNA encoding FVIII muteins of the invention may be provided to a patient in need thereof, concurrent with, or immediately after diagnosis.

The muteins, materials, compositions, and methods described herein are intended to be representative examples of the invention, and it will be understood that the scope of the invention is not limited by the scope of the examples. Those skilled in the art will recognize that the invention may be practiced with variations on the disclosed polypeptides, materials, compositions and methods, and such variations are regarded as within the ambit of the invention.

The following examples are presented to illustrate the invention described herein, but should not be construed as limiting the scope of the invention in any way.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

### Endocytosis of FVIII by Dendritic Cells

The effect of FVIII glycosylation on uptake by DCs in vitro was determined. Full-length rFVIII was first labeled for FACS analysis and then deglycosylated. For labeling of rFVIII for FACS analysis, 6 μg fluorescein isothiocyanate (FITC) in PBS (pH 9) was added to 100 μg deglycosylated FVIII and allowed to mix for 2 hours at 4° C. Unconjugated FITC was removed by dialysis using a 50K membrane in a solution of 20 mM HEPES, 150 mM NaCl, 2% sucrose, and 100 ppm Tween®-80 (polyethylene glycol sorbitan monooleate) at pH 7.5 for 2 hours at 4° C. FVIII concentration was quantified by Bradford assay and FVIII activity was determined by chromogenic assay. Labeled rFVIII was then enzymatically deglycosylated using endoglycosidase F1 (Endo-F1), which specifically cleaves N-linked oligosaccharides without denaturing the protein. rFVIII was incubated with Endo-F1 for 1 hour at 37° C. rFVIII was injected into a 50K membrane and dialyzed against a solution of 20 mM HEPES, 150 mM NaCl, 2% sucrose, and 100 ppm Tween®-80 at pH 9 for 2 hours at 4° C. Deglycosylation was confirmed by western blot analysis.

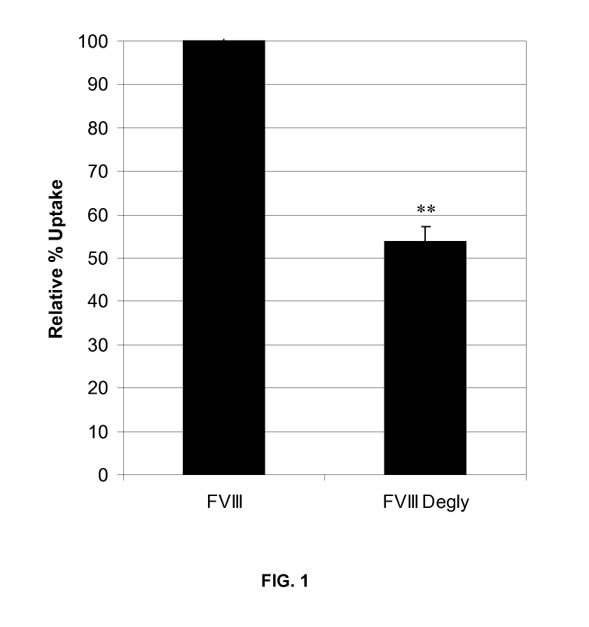

To generate dendritic cells (DC), adherent monocytes were cultured in RPMI 1640 media (Hyclone/Thermo Scientific, Logan, Utah) supplemented with 3% human AB serum, 20 ng/mL GM-CSF and 10 ng/mL IL-4 for 5 days. DC viability was confirmed by flow cytometry. All cells were cultured at 37° C. in humidified cell incubators with 5% CO₂ and 95% air. To determine the effect of rFVIII deglycosylation on uptake by DCs, DCs were incubated for 30 minutes with deglycosylated rFVIII, and after incubation, were analyzed for uptake of FVIII by the DCs by FACS. FIG. 1 shows that uptake of FVIII by DCs is significantly reduced following deglycosylation by Endo-F1. These results show that uptake of FVIII by DCs is dependent at least in part on N-glycosylation and further suggest that uptake is mediated by CD206 which recognizes N-linked non-capped oligosaccharides.

### Example 2

### Expression of FVIII Muteins in HKB11 Cells

A BDD FVIII and three muteins of this BDD FVIII were expressed in HKB11 cells. The BDD FVIII contained a deletion of all but 14 amino acids of the B-domain, such that the first 4 amino acids of the B-domain were linked to the 10 last residues of the B-domain. One BDD FVIII mutein contained a single substitution of glutamine for asparagine at position 239 (N239Q), another contained a single substitution of glutamine for asparagine at position 2118 (N2118Q) and the third contained both mutations (N239Q/N2118Q).

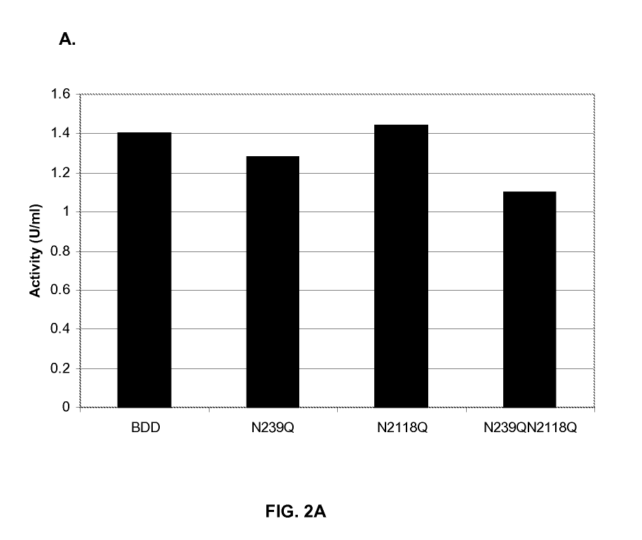

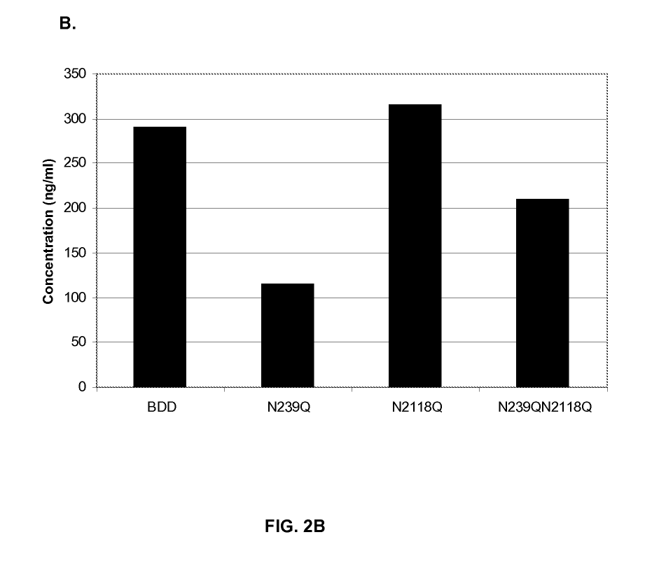

HKB11 cells were transiently transfected with BDD FVIII and BDD FVIII mutein expression plasmids using Lipofectamine™ 2000 (Invitrogen, Carlsbad, Calif.) according to the manufacturer's instructions. HKB11 cells were transiently transfected with BDD and BDD mutein plasmids, and supernatants from these cells were tested for FVIII activity by a chromogenic assay and for FVIII concentration by ELISA. The specific activity of the three muteins was found to be similar to the BDD which contained the respective glycosylation sites in unmutated form (FIG. 2A). The N2118Q mutein exhibited expression levels similar to BDD while N239Q and N239Q/N2118Q mutein expression levels were approximately 25% and 50% lower, respectively, than BDD (FIG. 2B). Accordingly, while yield of some muteins in this exemplary system was reduced, the muteins were nonetheless recovered in useful quantities.

### Example 3

### Reduced Uptake of FVIII Muteins by Dendritic Cells

Because uptake of FVIII by dendritic cells (DCs) is thought to be mediated by CD206 interaction with mannose-ending glycans on FVIII as described in Example 1, the capacity of DCs to take up the N239Q/N2118Q BDD mutein was tested. DCs were prepared as described above. DCs from two donors were pooled and then co-cultured with full-length rFVIII, BDD FVIII (described in Example 2), or the N239Q/N2118Q BDD mutein. Cells were co-cultured for 30 minutes in wells of a 96-well plate. The final volume per well was 100 μL and the final concentration of rFVIII, BDD, or mutein was 10 nM. The plate was then incubated for 30 minutes at 37° C. A parallel uptake assay was also performed at 4° C. as a control. Cells were pelleted by centrifugation of the plate at 300 g for 5 minutes at 4° C. Media were aspirated and cells were washed three times with ice-cold PBS/10 mM EDTA/0.01% Tween®-80. Cell pellets were then lysed by 25 μL per well of Cytobuster™ buffer (Novagen, Madisen, Wis.) with protease inhibitor for 15 minutes at 4° C. The plate was centrifuged for 10 minutes at 300 g before ELISA. For ELISA (American Diagnostica, Stamford, Conn.), cell extracts were diluted 1/25. Standard curves (80 to 1.25 μmolar) for FVIII and BDD were generated from recombinant proteins. ELISA was performed according to manufacturer's instruction.

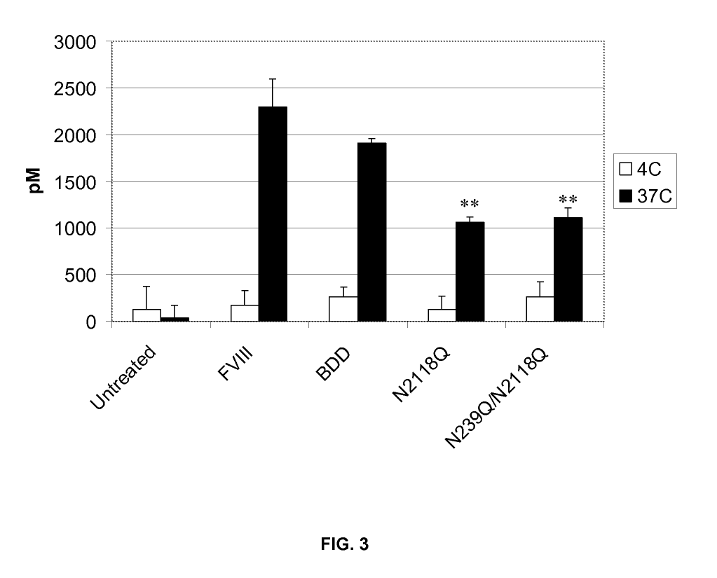

FIG. 3 shows that uptake of the N2118Q mutein and the N239Q/N2118Q FVIII mutein by DCs was significantly lower than that of rFVIII and BDD.

In a pharmacokinetic study of BDD 2118Q, the BDD N2118Q mutein was injected intravascularly at 0.05 mg/kg into male Sprague Dawley Rats (n=4). Blood samples were drawn at various time points, and the concentration of BDD N2118Q was measured by absorbance at 280 nm. The half-life of the single mutant in rats was 4.4±0.7 hours, similar to BDD.

### Example 4

### Reduced in vitro IFNγ Response of FVIII-Specific T-cell Clones

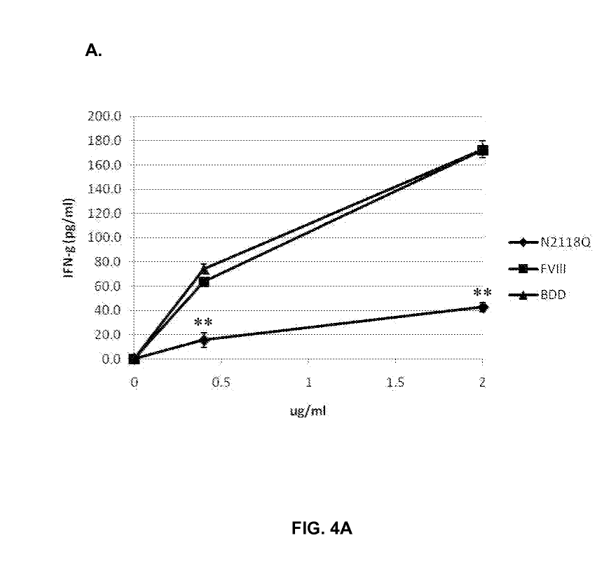

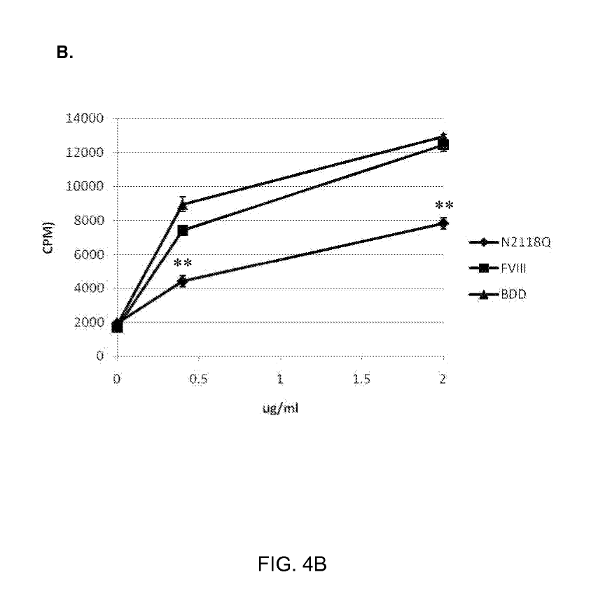

To test whether a reduction in uptake of N2118Q results in reduced T-cell activity against FVIII, secretion of IFNγ by FVIII-specific T-cell clone BO1-4 was tested (FIG. 4A). HLA-matched DCs were incubated for 24 hours with FVIII, BDD, or N2118Q for 24 hours at 37° C. in autologous plasma to enable uptake, processing, and presentation of each protein by DCs. DCs were then co-cultured with FVIII-specific T-cell clones (10:1 ratio of T-cells:DCs) for 24 hours at 37° C. Supernatant (50 μL) was then collected and diluted two-fold for measurement of IFNγ by enzyme-linked immunosorbent assay (ELISA).

### Example 5

### Reduced in vitro Proliferative Response of FVIII-Specific T-cell Clones

To test whether a reduction in uptake of N2118Q results in reduced T-cell proliferation in response to FVIII, secretion of IFNγ by FVIII-specific T-cell clone BO1-4 was tested (FIG. 4B). HLA-matched DCs were incubated for 24 hours with FVIII, BDD, or N2118Q for 24 hours at 37° C. in autologous plasma to enable uptake, processing, and presentation of each protein by DCs. DCs were then co-cultured with FVIII-specific T-cell clones (10:1 ratio of T-cells:DCs) at 37° C. At day 3, 20 μCi 3H-thymidine (thymidine) was added for an additional 36 hours. Cells were harvested and tested for thymidine incorporation.

FIGS. 4A and 4B show a significantly reduced IFNγ and proliferative response against N2118Q by BO1-4 T-cell clones. These data support the notion that a reduction in the uptake N2118Q by DCs results in a diminished capacity by DCs to present FVIII peptides to FVIII-specific T-cell clones.

All publications and patents mentioned in the above specification are incorporated herein by reference. Various modifications and variations of the described methods of the invention will be apparent to those skilled in the art without departing from the scope and spirit of the invention.

Although the invention has been described in connection with specific embodiments, it should be understood that the invention as claimed should not be unduly limited to such specific embodiments. Indeed, various modifications of the above-described modes for carrying out the invention which are obvious to those skilled in the field of biochemistry or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

1. A recombinant Factor VIII molecule comprising an amino acid sequence that has been modified by introducing one or more amino acid mutations within one or more naturally-occurring N-linked glycosylation site amino acid sequences wherein said mutation prevents the N-linked glycosylation site from being glycosylated.

2. The recombinant Factor VIII molecule of claim 1, wherein the N-linked glycosylation site amino acid sequences are selected from the group consisting of amino acid positions 41-43, 239-241, 582-584, 1810-1812, and 2118-2120 of a Factor VIII molecule.

3. The recombinant Factor VIII molecule of claim 2, wherein the amino acid positions are 239-241, 1810-1812, and 2118-2120.

4. The recombinant Factor VIII molecule of claim 2, wherein the one or more amino acid mutations comprise one or more amino acid mutations at position 239, position 1810, and position 2118.

5. The recombinant Factor VIII molecule of claim 2, wherein the mutations comprise mutations at positions 239 and 1810.

6. The recombinant Factor VIII molecule of claim 2, wherein the mutations comprise mutations at positions 239 and 2118.

7. The recombinant Factor VIII molecule of claim 2, wherein the mutations comprise mutations at positions 1810 and 2118.

8. The recombinant Factor VIII molecule of any of claim 1, wherein the mutation comprises a substitution.

9. The recombinant Factor VIII molecule of claim 8, wherein the substitution comprises the substitution of asparagine at position 239 with glutamine.

10. The recombinant Factor VIII molecule of claim 8, wherein the substitution comprises the substitution of asparagine at position 1810 with glutamine.

11. The recombinant Factor VIII molecule of claim 8, wherein the substitution comprises the substitution of asparagine at position 2118 with glutamine.

12. The recombinant Factor VIII molecule of claim 8, wherein the substitutions comprise the substitutions N239Q and N21180.

13. The recombinant Factor VIII molecule of any of claim 1, wherein the Factor VIII molecule is a B-domain deleted Factor VIII molecule.

14. An isolated nucleic acid that encodes the recombinant Factor VIII molecule of any of claims 1 to 13.

15. An expression vector comprising the nucleic acid of claim 14.

16. A glycosylation-competent host cell comprising the expression vector of claim 15.

17. A cell culture comprising the glycosylation-competent host cell of claim 16.

18. A pharmaceutical composition comprising the recombinant Factor VIII molecule of any of claims 1 to 13.

19. A composition according to claim 20 which is lyophilized for storage and can be reconstituted into a liquid for administration.

20. A method of treating a patient in need of Factor VIII therapy, comprising administering to said patient a therapeutically effective amount of the recombinant Factor VIII molecule of any of claims 1 to 13.

21. A method of treating a patient in need of Factor VIII therapy, comprising administering to said patient a therapeutically effective amount of the pharmaceutical composition of claim 18.

22. A method of treating a patient in need of Factor VIII therapy by gene therapy, comprising administering to the patient a composition comprising a therapeutic vector encoding a Factor VIII molecule.
